(19) United States
(12) Patent Application Publication
(10) Pub. No.: US 2005/0119564 A1
(43) Pub. Date: Jun. 2, 2005

(54) Pre-operative planning of implantations

(76) Inventors: Anders Rosholm, Hellerup (DK); Ulrik Olesen, Kobenhavn (DK); Niels Baadegaard, Virum (DK); Rolf Sanne, Helsingborg (SE)

Correspondence Address: HARNESS, DICKEY & PIERCE, P.L.C., P.O. BOX 8910, RESTON, VA, 20195, US

(21) Appl. No.: 10/722,526

(22) Filed: Nov. 28, 2003

(51) Int. Cl. A61B 5/05

(52) U.S. Cl. 600/429

(57) ABSTRACT

A method for positioning a digital template of a prosthesis based on computerized measurement of the width of the medullar space in an 2-dimensional projection image of the relevant bone into which the prosthesis is to be inserted, the method comprising detecting, preferably automatically, the edges of the medullar space, the edges being detected in a region of interest; determining a position of the template along, such as parallel to, the main bone orientation in such a manner that one or more points on the template and one or more other points in the image has a pre-defined geometrical relationship; and determining an orientation of the digital template and a position orthogonal to the main bone orientation such that the contours of the templates fit the detected edges of the medullar space.

The present invention relates to a method and a system to be used pre-operatively by surgeons to obtain guidance in the proper dimensions and model of implants to be surgically implanted in a living body. The method and system suggest the proper dimensions and model of a prosthesis, potentially based on user input defining anatomical landmarks in an image of the relevant body part, for example the hip. The image may be made by x-rays or other imaging techniques. The present invention relates in particular to to the estimation of appropriate prosthesis dimensions and model prior to the implantation of prosthesis into the body. The invention estimates the appropriate dimensions from an image of the relevant body part to be substituted with a prosthesis.

### BACKGROUND OF THE INVENTION

Presently, when preparing an implantation of for example a hip prosthesis, an appropriate model of the prosthesis and corresponding appropriate prosthsesis dimensions are estimated, typically by the surgeon responsible for the operation. The aim is prior to the operation to find a prosthesis that optimally fits the patient.

Prosthesis models exist as 2-dimensional templates, depicting the contours of the prostheses as they appear in a relevant projection (typically the AP projection).

The pre-determination of an appropriate prosthesis reduces the risk for the surgeon of having to insert a number of different prostheses during the operation before finding one that actually fits the bone dimensions of the patient.

Due to their distance from the film media (conventional film, CR cassettes or digital detectors) bones and other objects are subject to varying degrees of magnification when imaged by x-ray equipment. For hip x-rays, large people with much soft tissue tends to have the imaged bones enlarged more than smaller people. This magnification effect implies that the object size observed in an x-ray typically differs from the real physical size. The difference in observed and real size should be taken into account when finding appropriate prostheis models based on the matching of 2D templates with the image contents.

In prior art systems, the determination of an appropriate template is often made based on a visual evaluation of a conventional x-ray of the relevant body part.

Transparent 2-dimensional templates (printed acetates) of the possible prostheses are manually overlaid by the surgeon onto the x-ray in order to visually evaluate whether the prosthesis will fit the body part to which it shall be attached or inserted in. The 2-dimensional templates depict the contours of the corresponding prostheses calculated in the relevant projection direction of the x-ray.

When evaluating whether a template fits the bone part to which it shall be attached to or inserted in, the transparent templates are manually moved and rotated by the user such that a subjectively best fit between the template and the relevant bone part is achieved.

Prior to the template evaluation, a certain magnification of the bones is assumed and the matched templates are chosen in a correspondingly enlarged scale. Typically a magnification of 15-20% is assumed.

In addition to being relatively time consuming, the manual procedure for matching templates with the image contents has several drawbacks, including human operator imprecision and inter-operator variations.

The advent of digital (or digitized) x-rays has fundamentally changed the way that matching of templates may be carried out. The reading of x-rays is done from high-resolution computer screens and template matching using the transparent physical templates has effectively become impractical. Instead, the transparent physical templates have been substituted with digital versions of the templates that may be displayed as an overlay onto the screen, displaying beneath the digital x-ray of the relevant body part.

In the digital set-up with both digital x-rays and digital templates, a manual procedure quite similar to the previous “analogue” template matching procedure may be facilitated by enabling the operator (typically the surgeon) to load a digital template onto the screen and control its position and orientation using for example a computer mouse.

The correct scale of the digital templates is either estimated using the same ad-hoc assumptions as for conventional x-rays or through calibration of the digital image via the observed pixel size of an object in the image of known physical size. To achieve a correct magnification, estimated objects should preferably be placed in the same distance from the film media as the relevant bone.

The digital version of manually matching templates suffers from the same inconveniences as its analogue counterpart, i.e. it is prone to imprecision and operator-variability. The position and orientation of a digital template on the screen will further typically be controlled through mouse movements and mouse clicks, in which case the amount of time and mouse clicks used by the operator may be perceived as significantly inconvenient and inefficient. There is therefore a demand for software tools that may assist the operator and reduce the time and effort to perform the template matching.

### SUMMARY OF THE INVENTION

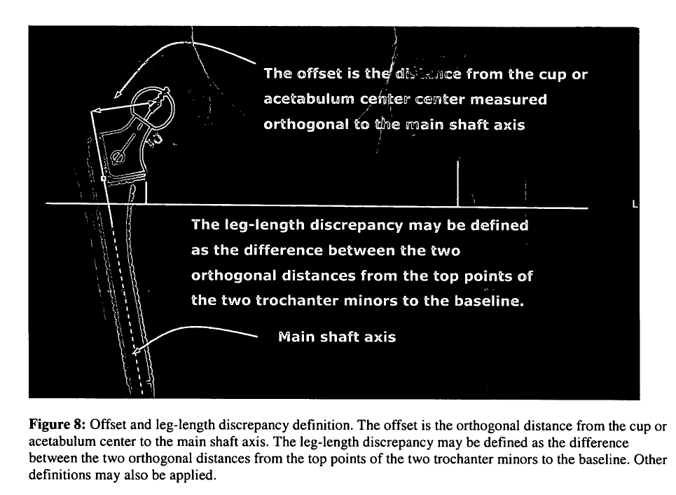

In a first aspect the present invent relates to a method for positioning a digital template of a prosthesis based on computerized measurement of the width of the medullar space in an 2-dimensional projection image of the relevant bone into which the prosthesis is to be inserted, the method comprising:
a. detecting, preferably automatically, the edges of the medullar space, the edges being detected in a region of interest.
b. determining a position of the template along, such as parallel to, the main bone orientation in such a manner that one or more points on the template and one or more other points in the image has a pre-defined geometrical relationship, an example of which is given in FIG. 8 illustrating that a certain point on a template is required to be located on a pre-defined line, thereby reducing the degrees of freedom for the template position by 1,
c. determining an orientation of the digital template and a position orthogonal to the main bone orientation such that the contours of the templates fit the detected edges of the medullar space.

In a second aspect the invention relates to a system for positioning a digital template of a prosthesis based on computerized measurement of the width of the medullar space in an 2-dimensional projection image of the relevant bone into which the prosthesis is to be inserted, the system being adapted by conventional computer and imaging means to:
a. detecting, preferably automatically, the edges of the medullar space, the edges being detected in a region of interest.
b. determining a position of the template along, such as parallel, the to the main bone orientation in such a manner that one or more points on the template and one or more other points in the image has a certain geometrical relationship, an example of which is given in FIG. 8 illustrating that a certain point on a shaft template is required to be located on a pre-defined line, thereby reducing the degrees of freedom for the template position by 1.
c. determining an orientation of the digital template and a position orthogonal to the main bone orientation such that the contours of the templates fit the detected edges of the medullar space.

The system according to the second aspect comprises preferably functionality so that it can perform one or more of the steps of the invention according to the first aspect.

The present invention meets the demand for a more efficient digital template matching procedure by suggesting an optimal size and position of a template based on detection of relevant anatomical characteristics found by image analysis.

The significant computational power of modern desktop computers facilitates that dedicated image analysis algorithms are applied to semi-automate the matching of a digital template with the image contents.

The present invention provides a novel method/system for assisting a surgeon (or operator) in determining an appropriate prosthesis prior to an implantation.

The invention involves, preferably, application of image analysis to locate relevant anatomical characteristics in the image and couples subsequently this information with the geometrical characteristics of a digital template. The image information and the template information are used to calculate an appropriate position and orientation of the template to suggest to the user.

In the following the present invention and in particular a preferred embodiment thereof is disclosed with reference to the accompanying figures.

### DESCRIPTION OF THE PREFERRED EMBODIMENT

The preferred embodiment of the system comprises a computer and a dedicated software program that allows the user to mark certain landmarks in an image of the hip, which enables the system to define an appropriate region of interest at the femoral shaft (for which the implantation is relevant). Having defined an appropriate region of interest, the system then performs an edge detection analysis of the femoral shaft and matches this information with the contour information of the digital template. It further suggests an optimal position and orientation based on an appropriate match to the detected edges. Other similar embodiments may concern prostheses for hand bones, knees or other bones.

Determination of Position, Orientation and Fit

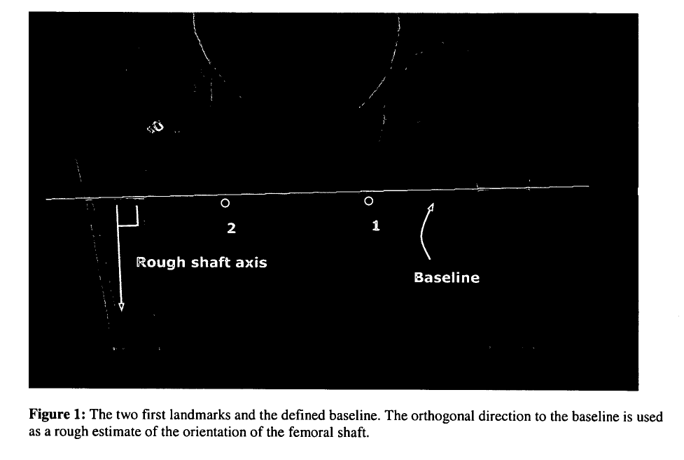

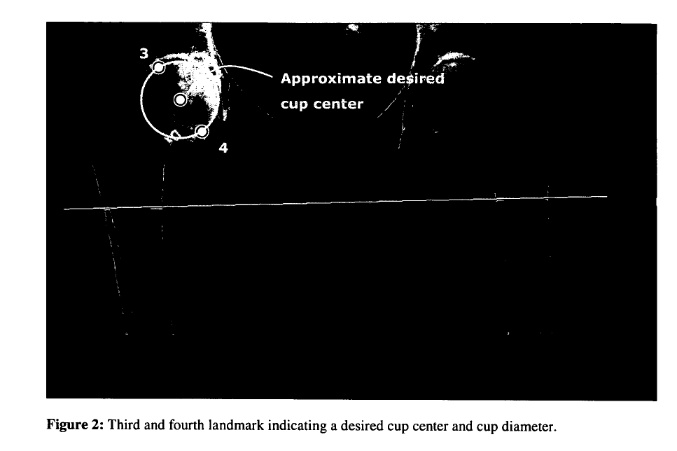

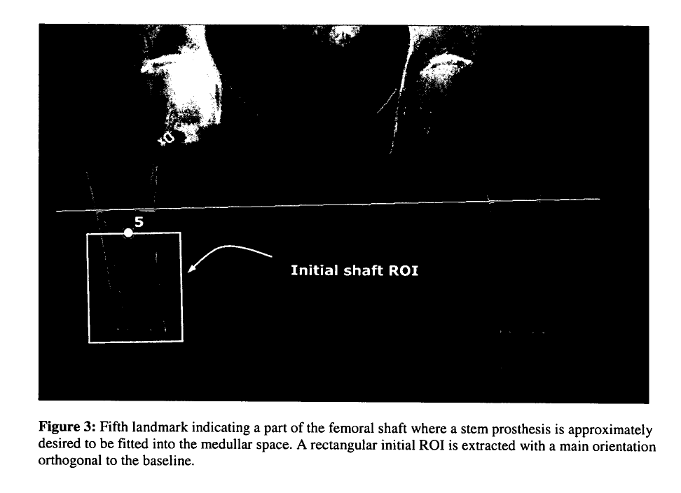

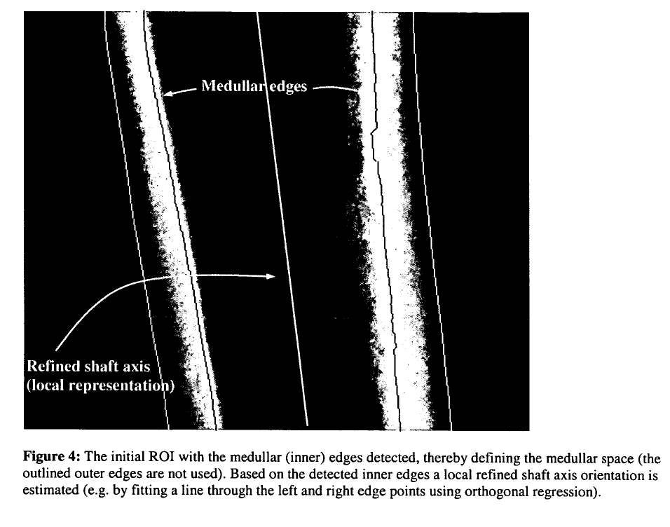

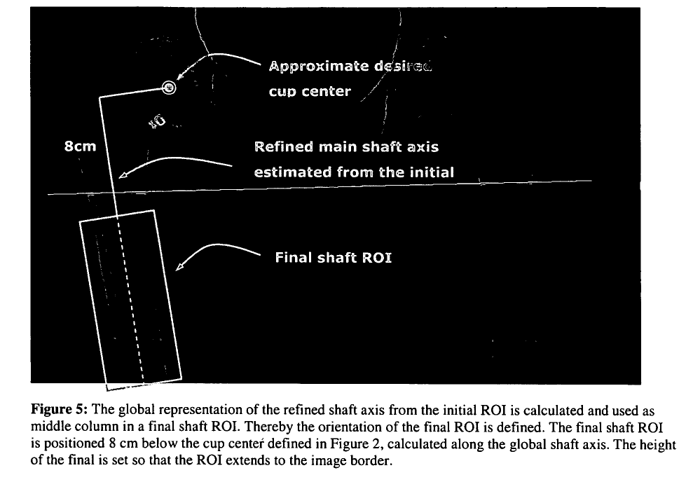

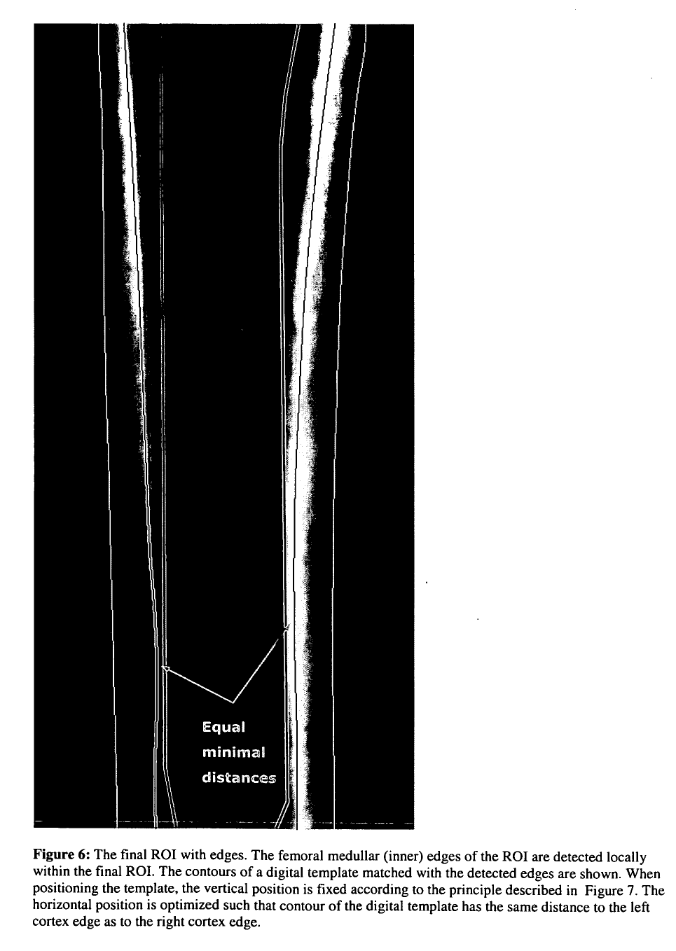

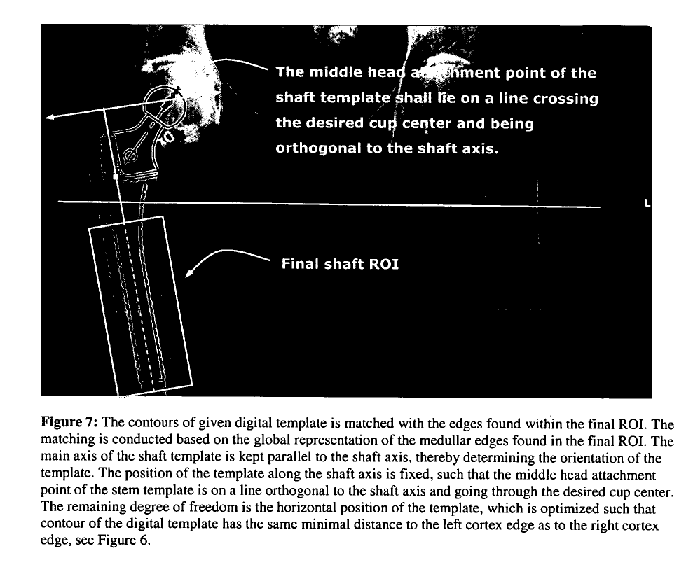

The following is carried out by the user and the system:
1. The user marks initially two landmarks (1 and 2 in FIG. 1) at the pelvis to define a line denoted the baseline. The baseline is subsequently used by the system to initially make a rough estimate of the main direction of the femoral shaft. The system then displays the baseline, see FIG. 1.
2. The user subsequently outlines, by two marked landmarks (3 and 4 in FIG. 2) on the acetabulum, an appropriate position and dimension of a cup prosthesis. From the two landmark points (3, 4) the system defines a circle with center at the midpoint of the two points (3, 4) and with a diameter equal to the distance between the two points, see FIG. 2. The center of the circle is later on used to define positions of involved Regions of Interest.
3. The user marks a point (5 in FIG. 3) indicating a part of the femoral shaft where a stem prosthesis approximately is desired to be fitted into the medullar space. The system extracts a rectangular Region of Interest (ROI) around the marked part of the femoral shaft. The direction orthogonal to the baseline is then used to define the main direction of the ROI, see FIG. 3.
4. The femoral medullar (inner) edges of the ROI are detected locally within the initial ROI, associating a medullar edge point with the point of maximum intensity of the corresponding cortex, see FIG. 4. Based on the detected inner edges a local refined shaft axis orientation is estimated (e.g. by fitting a line through the left and right edge points using orthogonal regression).
5. The global representation of the refined shaft axis from the initial ROI is calculated and used as middle column in a final shaft ROI. Thereby the orientation of the final ROI is defined. The top of the final shaft ROI is positioned 8 cm below the cup center defined in FIG. 2, calculated along the global shaft axis. The height of the final is set so that the ROI extends to the image border, see FIG. 5.
6. The femoral medullar (inner) edges of the ROI are detected locally within the final ROI, associating a medullar edge point with the point of maximum intensity of the corresponding cortex, see FIG. 6.
7. The contours of given digital template is matched with the edges found within the final ROI. The matching is conducted based on the global representation of the medullar edges found in the final ROI. The main axis of the shaft template is kept parallel to the shaft axis, thereby determining the orientation of the template. The position of the template along the shaft axis is fixed, such that the middle head attachment point of the stem template is on a line orthogonal to the shaft axis and going through the desired cup center, see FIG. 7. The remaining degree of freedom is the horizontal position of the template, which is optimized such that the contour of the digital template has the same minimal distance to the left cortex edge as to the right cortex edge, see FIG. 6 (by horizontal is meant orthogonal to the shaft axis).
8. The fitness measure is calculated as the minimal horizontal distance between template contours and the cortex edges (by horizontal is meant orthogonal to the shaft axis).
9. After determinating the optimal position of each template in a given set of available templates, the system suggests to the user the template with the best (lowest) fit.

The preferred embodiment described above deals at least with positioning a of digital template of a prosthesis The contours of the digital template is typically represented as x- and y-coordinates in a coordinate system with a pre-defined origo. The method is based on measurements of the width of the medullar space in an 2-dimensional projection image of the relevant bone into which the prosthesis is to be inserted, and the measurements are performed in a computerized manner in the sense that the for instance an image is digitized and loaded into a computer where the one or more ROI of interests are established typically by the before disclosed landmarking method.

Once a ROI is defined/selected, the edges of the medullar space within the ROI are detected, typically by applying an edge detecting algorithm which detects the edges preferably in a automated manner, that is without any interfering or guidance from a user of the method. The medullar edges in a cross-section of the bone is typically associated with the points of maximum intensity in the 2-dimensional projection image.

In the method, the main bone orientation is further estimated on the basis of the edges of the medullar space and/or the periosteal edges of the bone and the main bone orientation is in particular estimated by orthogonal regression through the medullar found edge points.

Following this step, a position of the template along, such as parallel to, the main bone orientation is determined. This is done in such a manner that a head attachment point of the shaft template lies on a line orthogonal to the bone axis going through a desired cup prosthesis center.

Once the position along the bone is determined, an orientation of the digital template and a position orthogonal to the main bone orientation are determined. It is noted, that the step of finding a position of the template along the main bone orientation and the step of determining the orientation of the digital template orthogonal to the main bone direction may be determined independently of each other resulting in that the steps does not have to performed in a specific order. The orientation and position are determined such that the contours of the templates best possibly fit the detected edges of the medullar space. A fit may either be represented by the minimal or the average distance between the template contours and the found edges. Also this step is preferably determined in an automated manner, and the fit between the template and the medullar edges is typically derived on the basis of minimizing the distances between the template contour and the edges.

The 2-dimensional projection image has preferably such a size that is covers at least part of the relevant bone and potentially extends to other body parts. As the method operates on a digital image many types of images may be utilized including an x-ray image.

One aim of the method is to suggest to the user an appropriate position a given template in an efficient manner requiring less user-interaction than with previous approaches. However, the method may also advantageously be applied to select a template from a plurality of templates. In this connection, the method is typically applied to a plurality of templates and a best fitting template, if present, is presented at its optimal position and rotation. A best fitting templates is characterized as the one having the smallest distance between the contours and the edges The templates are preferably stored in database from where they are loaded by the method. After execution of the method on the plurality of templates they are sorted according to the calculated fit for each template. Typically, a list of the sorted multiple templates is displayed to the user of the system.

Prior to determining the template position and orientation the method may assume a movement and/or orientation of the relevant bone as a result of the operation such that certain post-operational geometrical properties of the prosthesis is obtained. In particular, the relevant bone may be assumed moved and/or rotated during the operation such that a certain offset and/or leg-length discrepancy are obtained as a result of the operation, an example of which is disclosed in FIG. 8 illustrating offset and leg-length discrepancy.

The user may in particular specify that offset of the operated hip side should be the same as before the operation of the same as observed in the other side of the bone. The femoral shaft is then during the operation moved by the surgeon to a position that achieves this desired offset and the template position algorithm has to take this movement into account when matching the medullar edges of the shaft with the template contours. With respect to the leg-length discrepancy the user may likewise specify that it should be zero or at least be smaller than observed prior to the operation.

The method makes use of a hardware system adapted to perform the steps of the method disclosed. This hardware system comprises a number of physical entities typically comprising one or more scanners for digitizing images, pointing devices operatively connected to a computer with screens in such a manner that landmarks may be set and a visualization of the image with template as it appears from the accompanying figures.

Furthermore, the computer—or system in general—comprises calculation devices performing numerical calculations and storing devices storing the digital templates. Furthermore, the hardware system may advantageously make use of, comprise or further comprise a digital x-ray apparatus providing digitized x-ray images.

1. A method for positioning a digital template of a prosthesis based on computerized measurement of the width of the medullar space in an 2-dimensional projection image of the relevant bone into which the prosthesis is to be inserted, the method comprising:
a. detecting, preferably automatically, the edges of the medullar space, the edges being detected in a region of interest.
b. determining a position of the template along, such as parallel to, the main bone orientation in such a manner that one or more points on the template and one or more other points in the image has a pre-defined geometrical relationship
c. determining an orientation of the digital template and a position orthogonal to the main bone orientation such that the contours of the templates fit the detected edges of the medullar space.

2. A method according to claim 1 wherein the main bone orientation is estimated on the basis of the edges of the medullar space and/or the periostal edges of the bone.

3. A method according to claim 1 wherein the main bone orientation is estimated by a digital filtering technique.

4. A method according to claim 1 wherein the 2-dimensional projection image covers at least part of the relevant bone and potentially extends to other body parts.

5. A method according to claim 2 wherein the prosthesis is a femoral shaft prosthesis and the bone into which it shall be inserted is the femoral shaft.

6. A method according to claim 2 wherein the image may be derived as an x-ray image.

7. A method according to claim 2 wherein the medullar edges in a cross-section of the bone is associated with the points of maximum intensity in the 2-dimensional projection image.

8. A method according to claim 2 wherein the fit between the template and the medullar edges is derived by minimising the distances between the template contour and the edges.

9. A method according to claim 2 wherein the contours of the digital template is represented as x- and y-coordinates in a coordinate system with a pre-defined origo.

10. A method according to claim 2 wherein the method is applied to plurality of templates and a best fitting template, if present, is presented at its optimal position and rotation.

11. A method according to claim 10 wherein the plurality of templates is sorted according to the calculated fit for each template.

12. A method according to claim 11 where a list of the sorted multiple templates is displayed to the user of the system.

13. A method according to claim 5 wherein the system prior to determining the template position and orientation assumes a movement and/or orientation of the relevant bone as a result of the operation such that certain post-operational geometrical properties of the prosthesis is obtained.

14. A method according to claim 13 wherein the relevant bone is assumed moved and/or rotated during the operation such that a certain offset and/or leg-length discrepancy are obtained as a result of the operation, an example of which is disclosed in FIG. 8 illustrating offset and leg-length discrepancy.

15. A method according to claim 14 wherein the position and orientation of the femoral shaft prosthesis is determined such that the certain offset and/or leg-length discrepancy are obtained as a result of the operation.

16. A method according to claim 15 wherein the position and orientation of the femoral shaft prosthesis is determined such that the resulting offset for the operated femoral shaft is identical to the offset for the other femoral shaft.

17. A system for positioning a digital template of a prosthesis based on computerized measurement of the width of the medullar space in an 2-dimensional projection image of the relevant bone into which the prosthesis is to be inserted, the system being adapted by conventional computer and imaging means to:
a. detecting, preferably automatically, the edges of the medullar space, the edges being detected in a region of interest.
b. determining a position of the template along, such as parallel, the to the main bone orientation in such a manner that one or more points on the template and one or more other points in the image has a certain geometrical relationship.
c. determining an orientation of the digital template and a position orthogonal to the main bone orientation such that the contours of the templates fit the detected edges of the medullar space.

18. A system according to claim 17 wherein the main bone orientation is estimated by use of the system and on the basis of the edges of the medullar space and/or the periostal edges of the bone.

19. A system according to claim 17 wherein the main bone orientation is estimated by a digital filtering technique implemented in the system.

20. A system according to claim 17 wherein the 2-dimensional projection image covers at least part of the relevant bone and potentially extends to other body parts.

21. A system according to claim 17 wherein the prosthesis is a femoral shaft prosthesis and the bone into which it shall be inserted is the femoral shaft.

22. A system according to claim 17 wherein the comprising a digizing device, such as a scanner or a digital x-ray apparatus, to obtain the digitised image, the image may be derived as an x-ray image.

23. A system according to claim 18 wherein the medullar edges in a cross-section of the bone is associated with the points of maximum intensity in the 2-dimensional projection image.

24. A system according to claim 18 wherein the fit between the template and the medullar edges is derived by minimising the distances between the template contour and the edges.

25. A system according to claim 17, comprising a database storing the contours of the digital template, the digital template, and preferably the contours thereof, being represented as x- and y-coordinates in a coordinate system with a pre-defined origo.

26. A system according to claim 25, wherein a plurality of templates is stored and a best fitting template thereof, if present, is presented at its optimal position and rotation.

27. A system according to claim 26, wherein the plurality of templates is sorted according to the calculated fit for each template.

28. A system according to claim 17 wherein a list of the sorted multiple templates is displayed to the user of the system.

29. A system according to claim 17 wherein the system prior to determining the template position and orientation assumes a movement and/or orientation of the relevant bone as a result of the operation such that certain post-operational geometrical properties of the prosthesis is obtained.

30. A system according to claim 29 wherein the relevant bone is assumed moved and/or rotated during the operation such that a certain offset and/or leg-length discrepancy are obtained as a result of the operation, an example of which is disclosed in FIG. 8 illustrating offset and leg-length discrepancy.

31. A system according to claim 30 wherein the position and orientation of the femoral shaft prosthesis is determined such that the certain offset and/or leg-length discrepancy are obtained as a result of the operation.

32. A system according to claim 31 wherein the position and orientation of the femoral shaft prosthesis is determined such that the resulting offset for the operated femoral shaft is identical to the offset for the other femoral shaft.

33. A system according to claim 17, comprising at least one of the following devices: scanners for digitizing images, pointing devices operatively connected to a computer with one or more screens in such a manner that landmarks may be set and a visualization of the image with template, calculation devices performing numerical calculations and storing devices storing the digital templates and/or a digital x-ray apparatus providing digitized x-ray images.
